(19) United States
(12) Patent Application Publication
(10) Pub. No.: US 2018/0000681 A1
(43) Pub. Date: Jan. 4, 2018

(54) Method for Treatment of Internal Disorders Via Activation of Dermatomal Nervous Receptors

(71) Applicant: Gilbert Jaudy, Palm Desert, CA (US)

(72) Inventor: Gilbert Jaudy, Palm Desert, CA (US)

(21) Appl. No.: 15/200,241

(22) Filed: Jul. 1, 2016

(51) Int. Cl. A61H 1/02; A61N 1/04; A61F 7/00; A61B 5/0478; A61B 5/00; A61B 5/0484; A61N 1/36; A61H 23/00

(52) U.S. Cl. CPC A61H 1/0292; A61N 1/36014; A61N 1/0456; A61H 23/006; A61B 5/0478; A61B 5/4848; A61B 5/0484; A61F 7/00

(57) ABSTRACT

A system and method for treating neural lesions is disclosed. The inventive method comprises determining the location of a neural lesion in a patient and applying a selected modality to a dermatomal region on the patient to compensate for the existence of the neural lesion. The selected modality includes the implementation of a method or device to activate any number of receptors located in the skin of the patient. The modality implemented in the correct dermatomal region initiates a somatosensory signal to the brain which responds with a corresponding motor signal to the affected area. Proper implementation of the selected modality supports neuroplasticity and neurogenesis to create a permanent solution to overcome the neural lesion.

### FIELD OF THE INVENTION

The invention pertains generally to medical treatment and more specifically to a method of treating internal disorders through activation of dermatomal nervous receptors.

### BACKGROUND OF INVENTION

The medical industry is highly advanced. Medical issues with patients are currently treated with drugs and therapy. The treatment available is extremely effective in treating a patient's illness. For instance, extremely targeted pharmaceuticals have been developed to treat specific diseases with little to no side effects. Likewise, therapy can be effective in assisting a patient to deal with symptoms presented by an illness.

Current medical treatment, even though highly advanced, is limited in its perception of illnesses and treatment of illnesses. The standard model of medical treatment utilizes pharmaceuticals and therapy to lessen the symptoms of an illness. What is needed is a methodology of medical treatment that removes the underlying causes of an illness rather than mask the symptoms of an illness.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The invention is directed toward a method for treating a neural lesion comprising determining the existence of a neural lesion in a subject, selecting a dermatome on the subject, wherein the dermatome is innervated by a nerve connected to a neural pathway containing the neural lesion, and applying a therapeutic modality to the dermatome. The therapeutic modality is sufficient to activate neuroplasticity via synaptogenesis within collateral branches of the neural pathway.

In this method, the dermatome is selected from a group consisting of: C2, C3, C4, C5, T1, T2, T3, T4, T5, T6, T7, T8, T9, T10, T11, T12, L1, L2, L3, L4, L5, S1, S2, S3, S4, and S5. In addition, the therapeutic modality may be selected from any of the following: applying consistent pressure over a predetermined period of time with a reflex hammer; applying a rhythmic vibrational force over a predetermined period of time with a reflex hammer; stretching a portion of skin with a chiropractic instrument; applying consistent pressure over a predetermined period of time with a chiropractic instrument; applying a rhythmic vibrational force over a predetermined period of time with a chiropractic instrument; stretching a portion of skin with a chiropractic instrument; applying a rhythmic vibrational force over a predetermined period of time with a stylus of an electrical vibrational device; applying an object to a portion of skin of the subject, the object having a temperature greater than a temperature of the portion of skin of the subject; applying an object to a portion of skin of the subject, the object having a temperature less than a temperature of the portion of skin of the subject; poking a portion of skin of the subject with a needle; rolling a pinwheel on a portion of skin of the subject for a predetermined period of time; applying an electrical current to a portion of skin of the subject by means of a transcutaneous electrical nerve stimulation unit; applying two or more electrodes of a transcutaneous electrical nerve stimulation device to a portion of skin of the subject and generating a low intensity electrical impulse having a frequency of greater than 50 Hz from the two or more electrodes; performing a somatosensory evoked potential test.

The method may further comprise performing a somatosensory evoked potential (SEP) test on the subject prior to applying the therapeutic modality. The method may further comprise performing a second somatosensory evoked potential (SEP) test on the subject after applying the therapeutic modality to ensure efficacy of the therapeutic modality. The somatosensory evoked potential tests comprise the steps of: placing one or more stimulating electrodes on a chosen dermatome on the subject; placing one or more recording electrodes on the cranium of the subject; causing the one or more stimulating electrodes to generate an electrical impulse which is received by the one or more recording electrodes; and recording data received by the one or more recording electrodes.

Still other embodiments of the present invention will become readily apparent to those skilled in this art from the following description wherein there is shown and described the embodiments of this invention, simply by way of illustration of the best modes suited to carry out the invention. As it will be realized, the invention is capable of other different embodiments and its several details are capable of modifications in various obvious aspects all without departing from the scope of the invention. Accordingly, the drawing and descriptions will be regarded as illustrative in nature and not as restrictive.

### BRIEF DESCRIPTION OF THE DRAWINGS

Various exemplary embodiments of this invention will be described in detail, wherein like reference numerals refer to identical or similar components, with reference to the following figures, wherein:

FIG. 1 is a diagram of spinal nerves emanating from a spinal column;

FIG. 2 is a diagram of the human sympathetic nervous system;

FIG. 3 is a diagram of the human parasympathetic nervous system;

FIG. 4A is a diagram of dermatomes on the front of a human subject;

FIG. 4B is a diagram of dermatomes on the back of a human subject;

FIG. 5 is a diagram of a neural lesion in a neural pathway connected to the heart;

FIG. 6 is a diagram of a neural lesion in a neural pathway connected to the heart; and

FIG. 7 is a schematic of the method of the invention.

### DETAILED DESCRIPTION OF THE PREFERRED EMBODIMENT

The claimed subject matter is now described with reference to the drawings. In the following description, for purposes of explanation, numerous specific details are set forth in order to provide a thorough understanding of the claimed subject matter. It may be evident, however, that the claimed subject matter may be practiced with or without any combination of these specific details, without departing from the spirit and scope of this invention and the claims.

The inventive method is a segment of an overall systems approach to health care. This systems approach to health care can be envisioned as a pyramid with the systems approach as the tip of the pyramid. Underneath this systems approach to health care is systems integration diagnostics and treatment. Systems integration diagnostics and treatment is the concept that all human systems are connected (i.e. the endocrine system is connected to the nervous system is connected to the circulatory system). The diagnosis and treatment of one system cannot be performed in isolation of other systems. A physician should consider information and readings of all distinct systems when diagnosing and treating an issue presented by a patient.

Under the systems integration diagnostics and treatment are brain and organ remapping neuroplasticity treatments. These treatments are separate treatments which are utilized to effect brain remapping and neuroplasticity in a patient. Under the brain and organ remapping neuroplasticity treatments are organ remapping and brain remapping. The procedures falling under organ remapping are neurovisceral applications which are a specific set of applications which affect remapping and neuroplasticity related to the patient's organs. The procedures falling under brain remapping are brain based applications which are a specific set of applications which affect remapping and neuroplasticity in the patient's brain. The procedures of neurovisceral applications and brain based applications produce restoration of function through neurogenesis.

Underlying this restoration of process is the concept that the end product of these procedures is functional neurology. This is the concept that many problems and issues presented in a patient can be fixed through restoring the proper function of the nervous system. Underneath all of these is the concept of modern neuroscience treatment procedures and protocol. This concept is a shift in paradigm from the method of treating a patient's health problems in distinct and isolated segments. Under current medical philosophy a physician treats only the current issue presented by the patient at the moment of treatment. SIDT procedures and protocols in modern neuroscience treatment changes this to the philosophy that the patient cannot be treated in increments but instead that the physician must treat the entire patient, considering all systems of a patient when diagnosing and treating the health issue presented.

The inventive method is easiest to understand when viewed in light of knowledge of the anatomy and physiology of the human nervous system.

Central and Peripheral Nervous System

The human nervous system comprises the central nervous system and peripheral nervous system. The brain and the spinal cord make up the central nervous system. The largest mass of the brain is the cerebrum. The cerebrum comprises the outer gray matter that is the cortex and several deep nuclei that are split into functional groups. Within the cerebrum is the limbic cortex which is part of the limbic system. The hypothalamus, a part of the limbic system, is involved in regulating homeostasis. The hypothalamus is the region of the brain in charge of the autonomic nervous system and the endocrine system.

Peripheral Nervous System

The peripheral nervous system is everything that is not the central nervous system. The nerves of the peripheral nervous system can have structures called ganglia. A ganglion is a group of neuron cell bodies in the periphery. Ganglia can be categorized as either sensory ganglia or autonomic ganglia. The most common type of sensory ganglion is a dorsal (posterior) root ganglion. These ganglia are the cell bodies of neurons with axons that are sensory endings in the periphery, such as in the skin, and that extend into the CNS through the dorsal nerve root.

The other major category of ganglia are those of the autonomic nervous system, which is divided into the sympathetic and parasympathetic nervous systems. The sympathetic chain ganglia are a row of ganglia along the vertebral column that receive input from the lateral horn of the thoracic and upper lumbar spinal cord. Superior to the chain ganglia are three paravertebral ganglia in the cervical region. Three other autonomic ganglia that are related to the sympathetic chain are the prevertebral ganglia, which are located outside of the chain but have similar functions. They are referred to as prevertebral because they are anterior to the vertebral column. The neurons of these autonomic ganglia are multipolar in shape, with dendrites radiating out around the cell body where synapses from the spinal cord neurons are made. The neurons of the chain, paravertebral, and prevertebral ganglia then project to organs in the head and neck, thoracic, abdominal, and pelvic cavities to regulate the sympathetic aspect of homeostatic mechanisms.

In addition, there are a group of terminal ganglia that receive input from cranial nerves or sacral spinal nerves and are responsible for regulating the parasympathetic aspect of homeostatic mechanisms. These two sets of ganglia, sympathetic and parasympathetic, often project to the same organs—one input from the chain ganglia and one input from a terminal ganglion—to regulate the overall function of an organ. For example, the heart receives two inputs such as these; one increases heart rate, and the other decreases it. The terminal ganglia that receive input from cranial nerves are found in the head and neck, as well as the thoracic and upper abdominal cavities, whereas the terminal ganglia that receive sacral input are in the lower abdominal and pelvic cavities.

Nerves

Nerves are fibers of nerve cells extending from the central nervous system and are categorized as either cranial nerves extending from the brain or spinal nerves connected to the spinal cord. Cranial nerves are primarily responsible for the sensory and motor functions of the head and neck. Of the cranial nerves, the vagus nerve is responsible for contributing to homeostatic control of the organs of the thoracic and upper abdominal cavities. The vagus nerve primarily targets autonomic ganglia in the thoracic and upper abdominal cavities. The nerves connected to the spinal cord are the spinal nerves. All of the spinal nerves are combined sensory and motor axons that separate into two nerve roots. The sensory axons enter the spinal cord as the dorsal nerve root. The motor fibers, both somatic and autonomic, emerge as the ventral nerve root. The dorsal root ganglion for each nerve is an enlargement of the spinal nerve.

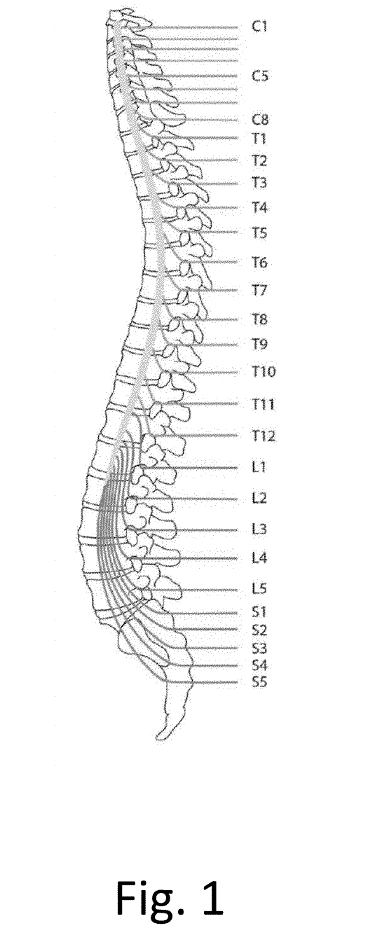

Referring to FIG. 1, there are 31 spinal nerves, named for the level of the spinal cord at which each one emerges. There are eight pairs of cervical nerves designated C1 to C8, twelve thoracic nerves designated T1 to T12, five pairs of lumbar nerves designated L1 to L5, five pairs of sacral nerves designated S1 to S5, and one pair of coccygeal nerves. The nerves are numbered from the superior to inferior positions, and each emerges from the vertebral column.

Spinal nerves extend outward from the vertebral column to enervate the periphery. The nerves in the periphery are not straight continuations of the spinal nerves, but rather the reorganization of the axons in those nerves to follow different courses. Axons from different spinal nerves will come together into a systemic nerve. This occurs at four places along the length of the vertebral column, each identified as a nerve plexus. Of the four nerve plexuses, two are found at the cervical level, one at the lumbar level, and one at the sacral level. The cervical plexus is composed of axons from spinal nerves C1 through C5 and branches into nerves in the posterior neck and head, as well as the phrenic nerve, which connects to the diaphragm at the base of the thoracic cavity. Spinal nerves C4 through T1 reorganize through the brachial plexus to give rise to the nerves of the arms. The lumbar plexus arises from all the lumbar spinal nerves and gives rise to nerves enervating the pelvic region and the anterior leg. The sacral plexus comes from the lower lumbar nerves L4 and L5 and the sacral nerves S1 to S4. Spinal nerves of the thoracic region, T2 through T11, are not part of the plexuses. These plexuses contain fibers that serve sensory functions or fibers that serve motor functions. Some fibers extend from cutaneous or other peripheral sensory surfaces and send action potentials into the central nervous system. Those are axons of sensory neurons in the dorsal root ganglia that enter the spinal cord through the dorsal nerve root. Other fibers are the axons of motor neurons of the anterior horn of the spinal cord, which emerge in the ventral nerve root and send action potentials to cause skeletal muscles to contract in their target regions.

Sensory Perception

Sensory receptors help us learn about the environment around us, or about the state of our internal environment. Stimuli are received and changed into the electrochemical signals of the nervous system. This occurs when a stimulus changes the cell membrane potential of a sensory neuron. The stimulus causes the sensory cell to produce an action potential that is relayed into the central nervous system, where it is integrated with other sensory information to become a conscious perception of that stimulus.

Sensory Receptors

Stimuli in the environment activate specialized receptor cells in the peripheral nervous system. Different types of stimuli are sensed by different types of receptor cells. Physical stimuli, such as pressure and vibration, as well as the sensation of sound and body position (balance), are interpreted through a mechanoreceptor. Another physical stimulus that has its own type of receptor is temperature, which is sensed through a thermoreceptor that is either sensitive to temperatures above (heat) or below (cold) normal body temperature. Physical touch on the skin can be subdivided into pressure, vibration, stretch, and hair-follicle position, on the basis of the type of mechanoreceptors that perceive these touch sensations. Other overlooked senses include temperature perception by thermoreceptors and pain perception by nociceptors.

Somatosensation is the group of sensory modalities that are associated with touch, proprioception, and interoception. These modalities include pressure, vibration, light touch, tickle, itch, temperature, pain, proprioception, and kinesthesia. Many of the somatosensory receptors are located in the skin, but receptors are also found in muscles, tendons, joint capsules, ligaments, and in the walls of visceral organs.

Two types of somatosensory signals that are transduced by free nerve endings are pain and temperature. These two modalities use thermoreceptors and nociceptors to transduce temperature and pain stimuli, respectively. Temperature receptors are stimulated when local temperatures differ from body temperature. Some thermoreceptors are sensitive to just cold and others to just heat. Nociception is the sensation of potentially damaging stimuli. Mechanical, chemical, or thermal stimuli beyond a set threshold will elicit painful sensations. Stressed or damaged tissues release chemicals that activate receptor proteins in the nociceptors.

Low frequency vibrations are sensed by mechanoreceptors called Merkel cells, also known as type I cutaneous mechanoreceptors. Merkel cells are located in the stratum basale of the epidermis. Deep pressure and vibration is transduced by lamellated (Pacinian) corpuscles, which are receptors with encapsulated endings found deep in the dermis, or subcutaneous tissue. Light touch is transduced by the encapsulated endings known as tactile (Meissner) corpuscles. Follicles are also wrapped in a plexus of nerve endings known as the hair follicle plexus, which detect the movement of hair at the surface of the skin. Stretching of the skin is transduced by stretch receptors known as bulbous corpuscles. Bulbous corpuscles are also known as Ruffini corpuscles, or type II cutaneous mechanoreceptors.

In summary: free nerve endings in the dermis respond to the stimuli of pain, temperature, and mechanical deformation; mechanoreceptors in the dermis respond to the stimulus of vibration; bulbous corpuscles in the dermis respond to the stimulus of stretch; tactile corpuscles in the dermis respond to the stimuli of light touch and vibration; lamellated corpuscles in the dermis respond to the stimuli of pressure and vibration; hair follicle plexi in the dermis respond to the stimulus of movement of hair.

Central Processing

A sensory pathway that carries peripheral sensations to the brain is referred to as an ascending pathway, or ascending tract. The various sensory modalities each follow specific pathways through the central nervous system. Tactile and other somatosensory stimuli activate receptors in the skin, muscles, tendons, and joints throughout the entire body. However, the somatosensory pathways are divided into two separate systems on the basis of the location of the receptor neurons. Somatosensory stimuli from below the neck pass along the sensory pathways of the spinal cord, whereas somatosensory stimuli from the head and neck travel through the cranial nerves—specifically, the trigeminal system.

The dorsal column system and the spinothalamic tract are two major pathways that bring sensory information to the brain. The axons in the dorsal column terminate in the nuclei of the medulla, where each synapses with the second neuron in their respective pathway. The second neuron in the system crosses the midline of the medulla and continues to ascend the brain stem. These axons terminate in the thalamus, where each synapses with the third neuron. The third neuron in the system projects its axons to the postcentral gyms of the cerebral cortex, where somatosensory stimuli are initially processed and the conscious perception of the stimulus occurs.

The spinothalamic tract also begins with neurons in a dorsal root ganglion. These neurons extend their axons to the dorsal horn, where they synapse with the second neuron in their respective pathway. Axons from these second neurons then ascend to the brain and enter the thalamus, where each synapses with the third neuron in its respective pathway. The neurons in the thalamus then project their axons to the spinothalamic tract, which synapses in the cerebral cortex. The dorsal column system is primarily responsible for touch sensations and proprioception, whereas the spinothalamic tract pathway is primarily responsible for pain and temperature sensations.

The sensory pathways of the trigeminal pathway each involve three successive neurons. These nuclei receive information like that carried by the dorsal column system, such as touch, pressure, vibration, and proprioception. In the thalamus, each axon synapses with the third neuron in its respective pathway. Axons from the third neuron then project from the thalamus to the primary somatosensory cortex of the cerebrum.

Autonomic Nervous System

The nervous system can be divided into two parts: the somatic nervous system and the autonomic nervous system. The somatic nervous system is associated with voluntary responses, and the autonomic nervous system is associated with involuntary responses, such as those related to homeostasis.

The autonomic nervous system regulates many of the internal organs through a balance of two aspects, or divisions. The autonomic nervous system is instrumental in homeostatic mechanisms in the body. The two divisions of the autonomic nervous system are the sympathetic division and the parasympathetic division. The sympathetic system is associated with the fight-or-flight response, and parasympathetic activity is referred to by the epithet of rest and digest. Homeostasis is the balance between the two systems. At each target effector, dual innervation determines activity. For example, the heart receives connections from both the sympathetic and parasympathetic divisions. The sympathetic system increases the heart rate and the parasympathetic system decreases the heart rate.

Sympathetic Division of the Autonomic Nervous System

To respond to a threat—to fight or to run away—the sympathetic system causes divergent effects as many different effector organs are activated together for a common purpose. More oxygen needs to be inhaled and delivered to skeletal muscle. The respiratory, cardiovascular, and musculoskeletal systems are all activated together. The digestive system shuts down so that blood is not absorbing nutrients when it should be delivering oxygen to skeletal muscles. To coordinate all these responses, the connections in the sympathetic system diverge from a limited region of the central nervous system to a wide array of ganglia that project to the many effector organs simultaneously. The complex set of structures that compose the output of the sympathetic system make it possible for these disparate effectors to come together in a coordinated, systemic change.

The sympathetic division of the autonomic nervous system influences the various organ systems of the body through connections emerging from the thoracic and upper lumbar spinal cord. The majority of ganglia of the sympathetic system belong to a network of sympathetic chain ganglia that runs alongside the vertebral column. The ganglia appear as a series of clusters of neurons linked by axonal bridges. The sympathetic nerve projects to the chain ganglion at the same level as the target effector (the organ, tissue, or gland to be innervated).

Collateral ganglia, also called prevertebral ganglia, are situated anterior to the vertebral column and receive inputs from splanchnic nerves as well as central sympathetic neurons. They are associated with controlling organs in the abdominal cavity, and are also considered part of the enteric nervous system. An axon from the central neuron that projects to a sympathetic ganglion is referred to as a preganglionic fiber or neuron, and represents the output from the central nervous system to the ganglion. A postganglionic fiber—the axon from a ganglionic neuron that projects to the target effector—represents the output of a ganglion that directly influences the organ.

As a response to a threat, the sympathetic system would increase heart rate and breathing rate and cause blood flow to the skeletal muscle to increase and blood flow to the digestive system to decrease. Sweat gland secretion should also increase as part of an integrated response. All of those physiological changes are going to be required to occur together to run away from a threat.

The neurons involved in the sympathetic system branch off in different pathways. A single preganglionic sympathetic neuron may have upwards of twenty targets that it innervates. For instance, when a fiber exits the spinal cord, one branch will synapse on a neuron in the adjacent chain ganglion. Other branches will innervate collateral ganglion. All of these branches means that one preganglionic neuron can influence different regions of the sympathetic system very broadly, by acting on widely distributed organs.

The sympathetic system causes changes in the body in preparation for fight or flight mechanisms of the body. The sympathetic system stimulates secretion of the sweat glands, constricts the blood vessels, dilates the pupils, decreases salivation, accelerates the heartbeat, relaxes the airways, inhibits digestion, stimulates secretion of epinephrine and norepinephrine, inhibits gut motility and secretions, and relaxes the urinary bladder.

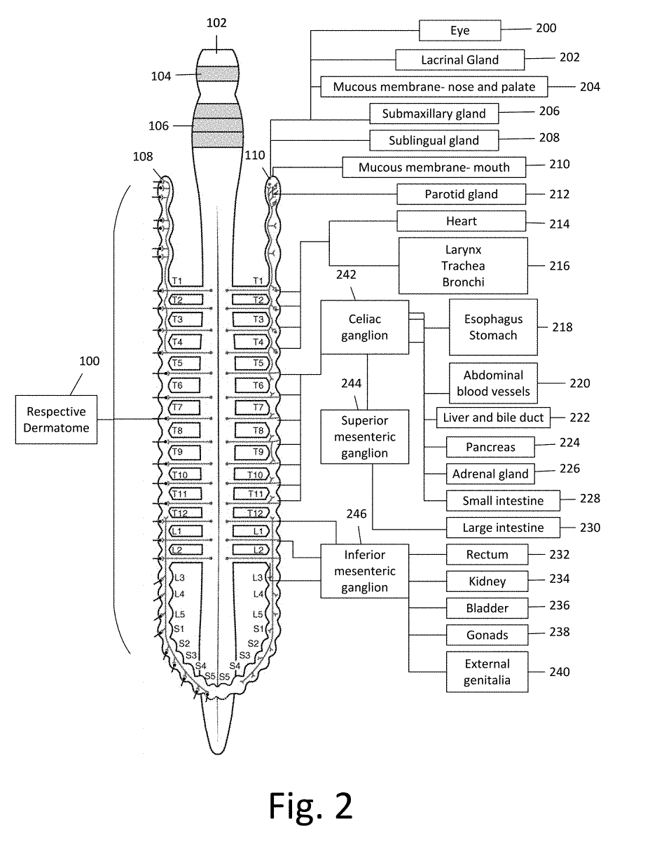

Referring FIG. 2, the connections of the human sympathetic nervous system is illustrated. The main part of the sympathetic nervous system is the spinal cord 102. At the higher end of the spinal cord 102 is the pons 104 and the medulla 106. Emanating from the spinal cord 102 are a plurality of spinal nerves. The spinal nerves connect into a right chain ganglia 108 and a left chain ganglia 110. Nerves emanating from the right chain ganglia 108 innervate separate sections of their respective dermatomes 100. Nerves emanating from the left chain ganglia 110 innervate separate organs. Nerves emanating from the superior cervical ganglia innervate the eye 200, lacrinal gland 202, mucous membrane—nose and palate 204, submaxillary gland 206, sublingual gland 208, mucous membrane—mouth 210, and parotid gland 210. The T1 through T4 spinal nerves innervate the heart 214 or larynx, trachea, or bronchi 216. The T5 through T11 spinal nerves combine into the celiac ganglion 242. Nerves emanating from the celiac ganglion 242 innervate the esophagus/stomach 216, the abdominal blood vessels 220, the liver and bile duct 222, the pancreas 224, the adrenal gland 226, and the small intestine 228. A nerve from the celiac ganglion 242 innervates the superior mesenteric ganglion 244, which then innervates the large intestine 230. The T12 through L3 spinal nerves combine into the inferior mesenteric ganglion 246. Nerves from the inferior mesenteric ganglion 246 innervate the rectum 232, kidney 234, bladder 236, gonads 238, and external genitalia 240.

Parasympathetic Division of the Autonomic Nervous System

The connections of the parasympathetic division are similar to the general layout of the sympathetic division. Parasympathetic preganglionic fibers primarily influence the heart, bronchi, and esophagus in the thoracic cavity and the stomach, liver, pancreas, gall bladder, and small intestine of the abdominal cavity. The postganglionic fibers from the ganglia activated by the vagus nerve are often incorporated into the structure of the organ, such as the mesenteric plexus of the digestive tract organs and the intramural ganglia.

The autonomic nervous system is important for homeostasis because its two divisions compete at the target effector. The balance of homeostasis is attributable to the competing inputs from the sympathetic and parasympathetic divisions. Organ systems are balanced between the input from the sympathetic and parasympathetic divisions. When something upsets that balance, the homeostatic mechanisms strive to return it to its regular state. For each organ system, there may be more of a sympathetic or parasympathetic tendency to the resting state, which is known as the autonomic tone of the system.

The parasympathetic system causes changes in the human body to calm the fight or flight mechanism. The parasympathetic system slows the heartbeat, constricts the airways, stimulates digestion, stimulates intestine activity, stimulates exocrine secretions, stimulates the gallbladder to excrete bile, stimulates gut motility, stimulates urinary bladder contraction, constricts the pupil, stimulates secretion from the lachrymal gland, stimulates salivation, and stimulates sexual arousal.

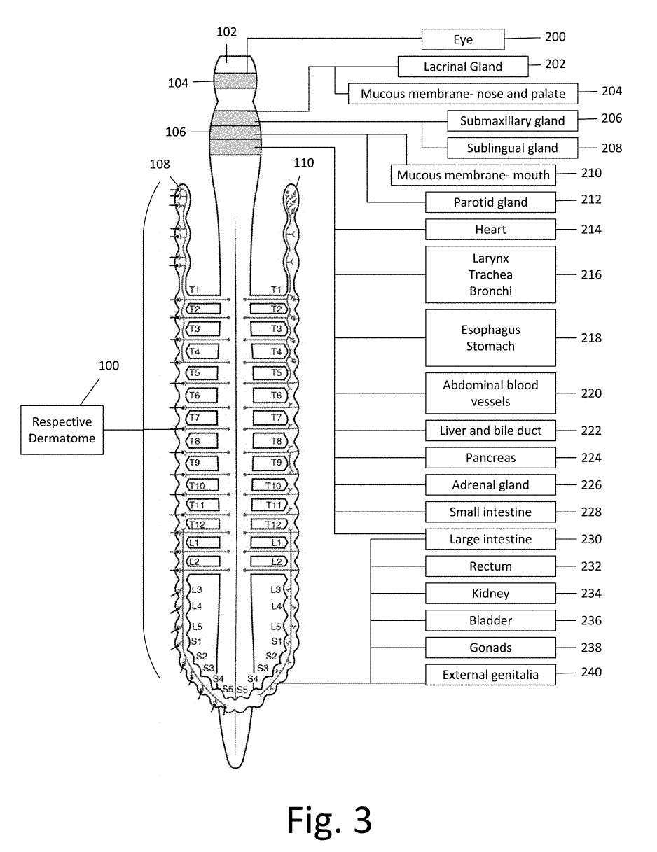

Referring to FIG. 3, the human parasympathetic system is illustrated. Cranial nerve III emanates from the Eddinger-Westphal nucleus in the pons 104, passes through the ciliary ganglion, and innervates the eye 200. Cranial nerve VII emanates from the super salivatory nucleus in the medulla 106 and innervates the pterygopalatine ganglion and submandibular ganglion. Nerves from the pterygopalatine ganglion innervate the lacrinal gland 202 and mucous membrane—nose and palate 204. Nerves from the submandibular ganglion innervate the submaxillary gland 206 and sublingual gland 208. Cranial nerve IX emanates from the inferior salivatory nucleus in the medulla 106 and innvervates the octic ganglion. Nerves from the octic ganglion innervate the mucous membrane—mouth 210 and parotid gland 212. Cranial nerve X emanates from the dorsal nucleus of the vagus nucleus ambigus in the medulla 106 and innervates the heart 214, the larynx, trachea, and bronchi 216, the esophagus/stomach 218, the abdominal blood vessels 220, the liver and bile duct 222, the pancreas 224, the adrenal gland 226, the small intestine 228, and the large intestine 230. A nerve from the S4 level innervates the large intestine 230, rectum 232, kidney 234, bladder 236, gonads 238, and external genitalia 240.

The nervous system comprises the brain and nerves that extend throughout the body outward from the brain. The main nerves travel down the spinal cord. Nerves branch off from the spinal cord. The spinal cord has several vertebra segmenting the spinal cord, as shown by FIG. 1. From the top of the spinal cord are the cervical vertebrae which are designated as C1, C2, C3, C4, C5, C6, C7, and C8. Connected to these are the thoracic vertebrae which are designated as T1, T2, T3, T4, T5, T6, T7, T8, T9, T10, T11, and T12. Connected to these are the lumbar vertebrae which are designated as L1, L2, L3, L4, and L5. Connected to these are the sacral vertebrae which are designated as S1, S2, S3, S4, and S5.

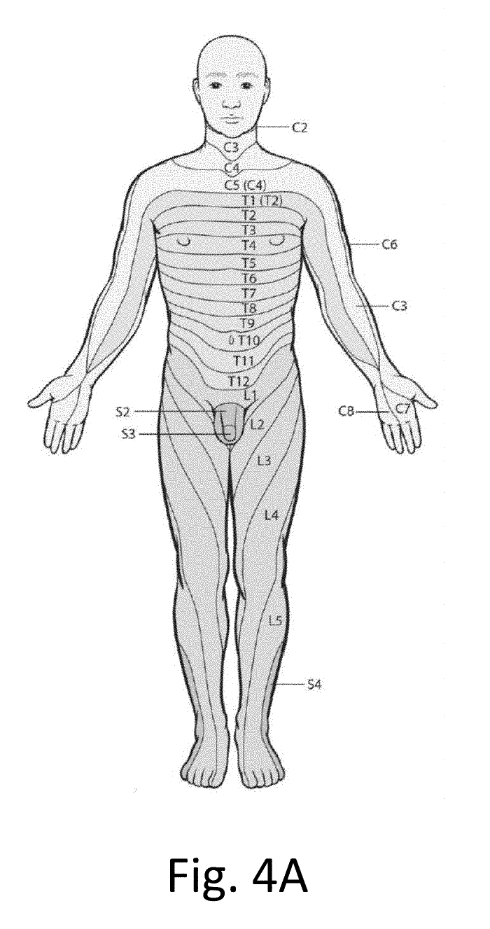

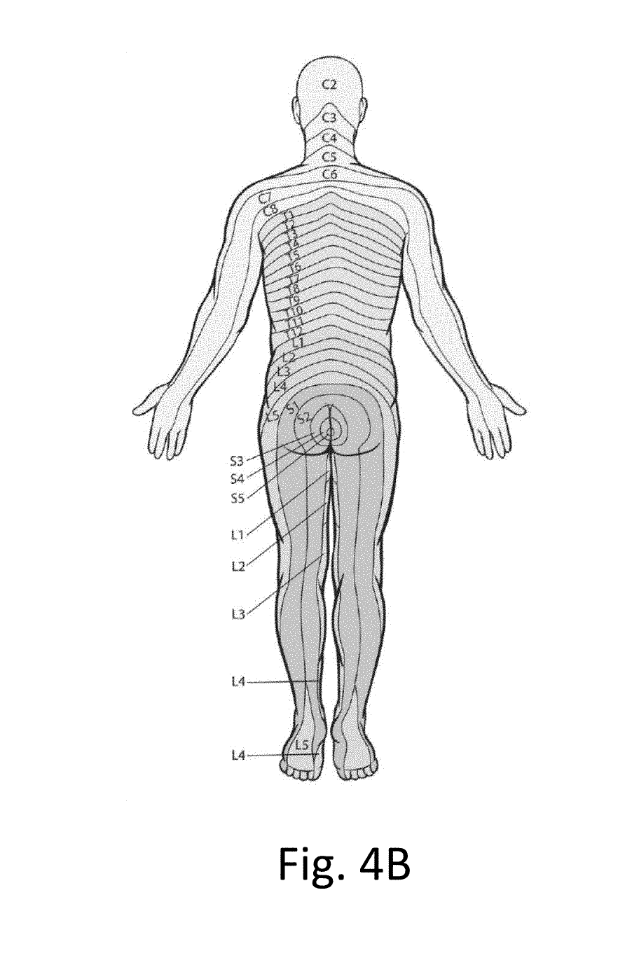

The nerves which branch out from the spine at each vertebra innervate separate organs within the body. Additionally, the nerves branching out from the spine at each vertebra also innervate a separate sections or areas of the skin, known as dermatomes. This is known as dermatomal segmentation. The location of dermatomes 100 is illustrated in FIG. 4A and FIG. 4B. Nerves from C2 vertebra innervate the face and top of the back of the head. Nerves from the C3 vertebra innervate the neck and portions of the arm. Nerves from the C4 vertebra innervate the collar bone area and back of the neck. Nerves from the C5 vertebra innervate the upper chest and base of the back of the neck. Nerves from the C6 vertebra innervate the upper back and portions of the arm. Nerves from the C7 vertebra innervate the back, portions of the arm, and fingers. Nerves from the C8 vertebra innervate the back, portions of the arm, and fingers. Nerves from the T1 vertebra innervate the back, the chest, and portions of the arm. Nerves from the T2 vertebra innervate the back, the chest, and portions of the arm. Nerves from the T3 vertebra innervate the back and the chest. Nerves from the T4 vertebra innervate the back and the chest. Nerves from the T5 vertebra innervate the mid-back and the mid-chest. Nerves from the T6 vertebra innervate the mid-back and the upper abdomen. Nerves from the T7 vertebra innervate the mid-back and the upper abdomen. Nerves from the T8 vertebra innervate the mid-back and the mid-abdomen. Nerves from the T9 vertebra innervate the mid-back and the mid-abdomen. Nerves from the T10 vertebra innervate the mid-back and the mid-abdomen. Nerves from the T11 vertebra innervate the lower-back and the lower-abdomen. Nerves from the T12 vertebra innervate the lower-back and the lower-abdomen. Nerves from the L1 vertebra innervate the upper pelvis and lower back. Nerves from the L2 vertebra innervate the upper legs and lower back. Nerves from the L3 vertebra innervate the upper legs and lower back. Nerves from the L4 vertebra innervate the legs and lower back. Nerves from the L5 vertebra innervate the legs and lower back. Nerves from the S1 vertebra innervate the buttocks and legs. Nerves from the S2 vertebra innervate the buttocks, genitals, and legs. Nerves from the S3 vertebra innervate the buttocks and genitals. Nerves from the S4 innervate the buttocks. Nerves from the S5 vertebra innervate the buttocks.

Neuroplasticity and Synaptogenesis

Neuroplasticity, also known as brain plasticity, is the change of the central nervous system. Neuroplastic change can occur at small scales, such as physical changes to individual neurons, or at whole-brain scales, such as cortical remapping in response to injury. Behavior, environmental stimuli, thought, and emotions may also cause neuroplastic change.

One component of neuroplasticity is synaptogenesis. Synaptogenesis is the formation of synapses between neurons in the nervous system. Activation of N-methyl-D-aspartate (NMDA) receptor in neurons of the central nervous system initiates synaptogenesis through activation of downstream products. The heightened level of NMDA receptor activity during development allows for increased influx of calcium, which acts as a secondary signal. Synaptic signaling during synaptogenesis is not only activity-dependent, but is also dependent on the environment in which the neurons are located.

The special structure found in the central nervous system that allows for multiple inputs is the dendritic spine, the site of excitatory synapses. This morphological dynamism is due to the specific regulation of the actin cytoskeleton, which in turn allows for regulation of synapse formation. Dendritic spines exhibit three main morphologies: filopodia, thin spines, and mushroom spines. The filopodia play a role in synaptogenesis through initiation of contact with axons of other neurons. Filopodia of new neurons tend to associate with multiple synapsed axons, while the filopodia of mature neurons tend to sites devoid of other partners. The dynamism of spines allows for the conversion of filopodia into the mushroom spines that are the primary sites of glutamate receptors and synaptic transmission.

Method of the Invention

Through a comprehensive neurological examination (after an extensive case history and patient/doctor consultation), regions of the nervous system that have become dysfunctional are identified. Also targeted are neurological pathways that are well intact (viable), which connect with dysfunctional regions. These viable pathways are then manipulated (non-surgically and non-pharmaceutically) through various modalities that utilize various myotomal and dermatomal regions to strengthen connections with faulty regions, causing neuroplasticity and regulation and normalization of function.

Using these methods, the organ systems, neurological pathways that have become dysfunctional, and brain regions that have become dysfunctional are targeted and through various non-invasive procedures, electrochemical signaling between brain regions and between regions of the brain and the body is regulated and normalized.

By regulating and normalizing the function of the brain and neural pathways and regulating and normalizing organ function and metabolism, symptoms and conditions are resolved in an overwhelming number of patients with a wide variety of conditions, including breathing disorders, regardless of the underlying mechanism (however, the underlying mechanisms must be first targeted through extensive and comprehensive testing and case history analysis).

The process begins when a patient is examined by a physician. First, the patient provides information to the physician. The physician can obtain information from the patient through a number of means, such as by presenting a questionnaire to the patient. The physician determines the location of the anatomical lesion in the nervous system of the patient. The physician gets the history of the patient, including the onset of the symptoms complained of, the chronology of the symptoms presented, the quality of the patient's health the severity and effect of the symptoms on the activities of the patient's life, any previous treatment that the patient has undergone, medical records and imaging records, and the overall health goals of the patient.

The physician also obtains the past health history of the patient, including information related to serious illnesses, hospitalizations and surgeries, any general trauma or injury, whether the patient is in menopause or is pregnant, if the patient is taking medication, whether the patient has allergies, any past diagnostic imaging such as x-ray, MRI, CAT-Scan, Doppler, or MRA, prior physician care, and the results of past physical or neurological exams.

The physician also obtains family and personal health history including family health problems, the patient's living and environment situation, the patient's amount of regular exercise, the patient's interests, diet, sleep pattern, bowel habits, urinary habits, stress factors, and emotional support system.

The physician also performs a general review of the systems of the patient. The physician reviews the general status of the patient's systems, including weight, and any weakness, fatigue, fever, sweats, chills, anorexia, or insomnia experienced by the patient. The physician also reviews other systems of the patient including the patient's skin, head, eyes, ears, nose, sinuses, throat and mouth, neck, and breasts. The physician also reviews the respiratory, cardiovascular, gastrointestinal, and genitourinary systems. The physician also reviews the female/male sexual organs and endocrine, musculoskeletal, neurological, hematologic, and psychiatric systems of the patient.

After obtaining all of the prior information from the patient the physician performs a comprehensive neurological examination of the patient. The neurological examination includes reviewing the emotional attitude, orientation, attention span, speech, memory, and cognition of the patient. The physician next reviews the patient's vitals and reflexes, such as deep tendon reflexes and pathological reflexes. The physician next performs a sensory exam of the patient, determines muscle strength of the patient, performs orthopedic examinations of the patient, and performs a palpation examination of the patient. The physician also performs a head and neck examination of the patient. This includes an otoscopic exam, opthalamascopic exam, chest exam, abdominal exam, and vascular exam.

The physician performs a cranial nerve exam of the patient, including reviewing: Blepherospasticity/Clonus; Olfactory Perception; Rapid Eye Movement; Optic Alignment; Hyper/Hypo; Lidlag/Ptosis; Pupil Size; Corectasia; Cormiosis; Pupillary Reflex; Optokinetic Tape; Parietal Pursuit; Refixation Saccade; Cerebellar Termination; Cardinal Gazes; Hypometria; Hypermetria; Convergence; Exophoria/Esophoria; Cover/Uncover; Facial Paresis; Voluntary motion; Lacrimal function (eyes/mouth); Weber's; Rhinne's (Mastoid); Air Cond; Palatal Paresis; Gag Reflex; Swallowing; Head Rotation; Shoulder Shrug; Hypoglossal; and Corneal Reflex.

The physician performs celebellar and cortical testing of the patient, including reviewing: Titubation; Romberg's; Direction of sway; Piano; Supination/Pronation; Finger to Nose; Heel to Toe Walk; Direction of sway; Past Pointing; Heel Down Shin; Heel Tap; Rapid Movement of Hands; Rapid Movement of Thumb/Index; Rapid Movement of Feet; Alternating Sup Pro; Two Point Disc.; Graphesthesia; Joint Position Sense; Heel Walk; and Toe Walk.

The physician performs orthopedic examination of the patient, including reviewing: lower left leg; lower right leg; straight leg raise; bilateral leg raise; soto hall, leg drop; Braggard's; Bilateral Leg Lowering; Goldthwait's; Lasegue's Sign; Well Leg Raising; Kemp's; Ely's; and fabere Patrick's.

The physician performs posture analysis of the patient including reviewing: head tilt (roll); head rotation (yaw); head flexion (positive pitch); head extension (negative pitch); high shoulder; high hip; wt. bearing (center of pressure); deviation; ellipse (R anterior-L posterior/L anterior-R posterior); gait; and arm swing.

The physician then performs a somatosensory evoked potential (SEP) test. An SEP test studies the relay of body sensations to the brain and how the brain receives those sensations. A stimulating electrode is placed on the patient's arm or leg. The stimulating electrode then generates an electrical signal. Recording electrodes are placed on the patient's head and/or spine. The information received from these electrodes can be utilized to diagnose the patient's issue. The SEP test evaluates the health of a patient's peripheral nerves and spinal cord. The SEP test also tests how the patient's spinal cord and/or brain transmits information about body sensations through the patient's peripheral nerves. The SEP test can localize a “signal blockage” in the patient's peripheral nerves, brain, or spinal cord. When recording SEPs, the physician may seek to study peripheral, spinal, brainstem, and early cortical SEPs during the same run. Recording electrodes placed on the scalp pick up both SEPs generated in the cortex and thalamocortical fibers (which are picked up as near-field responses located in restricted areas) and far-field positivities reflecting the evoked activity generated in peripheral, spinal and brainstem somatosensory fibers. The results of the SEP test can be used to determine and define an anatomical level of impairment along a neural pathway or otherwise determine the existence of silent lesions.

In the inventive method the physician may begin by performing a series of SEP tests on the patient. In each instance, the physician may place the stimulating electrodes on a separate dermatome of the patient. The physician then causes the stimulating electrodes to generate an electrical signal which is recorded by the recording electrodes on the patient's scalp. After ensuring that sufficient information is received for the first dermatome, the physician then places the stimulating electrodes on another dermatome and repeats the process. The physician repeats this testing process for all dermatomes to ensure that all neural lesions are located.

Through the results obtained in the testing and medical history, the physician diagnoses the cause of the symptoms. The physician is able to determine the location of the lesion in the nervous system of the patient.

The method of the invention provides for treating neurological lesions by instructing the neurological pathways to bypass lesions in the neurological pathway and strengthen connections through synaptogenesis of collateral branches of the neurological pathway. These connections are strengthened by applying a physical application to a chosen dermatome. The process may be utilized in the following ways:

The dermatome for C1 can be used to strengthen connections to the coronary, myocardial, and intestinal systems. In this instance the location of the neurological lesion is primarily in the brainstem or enteric nervous system. A physician applies a chosen modality to activate the C1 dermatome in this instance.

The dermatome for C2 can be used to strengthen connections to the respiratory system and the kidneys. In this instance the location of the neurological lesion is primarily in the brainstem or enteric nervous system. A physician applies a chosen modality to activate the C2 dermatome in this instance.

The dermatome for C3 can be used to strengthen connections to the gallbladder, gastric, and ileocecal systems. In this instance the location of the neurological lesion is primarily in the celiac ganglion, enteric nervous system or brainstem. A physician applies a chosen modality to activate the C3 dermatome in this instance.

The dermatome for C4 can be used to strengthen connections to the pancreas or cecum. In this instance the location of the neurological lesion is primarily in the celiac ganglion, superior mesenteric ganglion, or enteric nervous system. A physician applies a chosen modality to activate the C4 dermatome in this instance.

The dermatome for C5 can be used to strengthen connections to the spleen and glandular system. In this instance the location of the neurological lesion is primarily in the hemispheric microglia or celiac ganglion. A physician applies a chosen modality to activate the C5 dermatome in this instance.

The dermatome for C6 can be used to strengthen connections to the liver and colon. In this instance the location of the neurological lesion is primarily in the nucleus solitarius brainstem or liver ganglion. A physician applies a chosen modality to activate the C6 dermatome in this instance.

The dermatome for C7 can be used to strengthen connections to the adrenals and gonads. In this instance the location of the neurological lesion is primarily in the hypothalamus or pituitary axis. A physician applies a chosen modality to activate the C7 dermatome in this instance.

The dermatome for C8 can be used to strengthen connections to the pinkie finger. In this instance the location of the neurological lesion is primarily in the C8 nerve root. A physician applies a chosen modality to activate the C8 dermatome in this instance.

The dermatome for T1 can be used to strengthen connections to the coronary system. In this instance the location of the neurological lesion is primarily in the brainstem or enteric nervous system. A physician applies a chosen modality to activate the T1 dermatome in this instance.

The dermatome for T2 can be used to strengthen connections to the myocardial system. In this instance the location of the neurological lesion is primarily in the brainstem or enteric nervous system. A physician applies a chosen modality to activate the T2 dermatome in this instance.

The dermatome for T3 can be used to strengthen connections to the respiratory system. In this instance the location of the neurological lesion is primarily in the brainstem or enteric nervous system. A physician applies a chosen modality to activate the T3 dermatome in this instance.

The dermatome for T4 can be used to strengthen connections to the gallbladder. In this instance the location of the neurological lesion is primarily in the celiac ganglion, enteric nervous system, or brainstem. A physician applies a chosen modality to activate the T4 dermatome in this instance.

The dermatome for T5 can be used to strengthen connections to the gastric system. In this instance the location of the neurological lesion is primarily in the celiac ganglion, enteric nervous system, or brainstem. A physician applies a chosen modality to activate the T5 dermatome in this instance.

The dermatome for T6 can be used to strengthen connections to the pancreas. In this instance the location of the neurological lesion is primarily in the celiac ganglion, superior mesenteric ganglion, or enteric nervous system. A physician applies a chosen modality to activate the T6 dermatome in this instance.

The dermatome for T7 can be used to strengthen connections to the spleen. In this instance the location of the neurological lesion is primarily in the hemispheric microglia or celiac ganglion. A physician applies a chosen modality to activate the T7 dermatome in this instance.

The dermatome for T8 can be used to strengthen connections to the liver. In this instance the location of the neurological lesion is primarily in the nucleus solitarius brainstem or liver ganglia. A physician applies a chosen modality to activate the T8 dermatome in this instance.

The dermatome for T9 can be used to strengthen connections to the adrenal system. In this instance the location of the neurological lesion is primarily in the hypothalamus or pituitary axis. A physician applies a chosen modality to activate the T9 dermatome in this instance.

The dermatome for T10 can be used to strengthen connections to the intestinal system. In this instance the location of the neurological lesion is primarily in the brainstem or enteric nervous system. A physician applies a chosen modality to activate the T10 dermatome in this instance.

The dermatome for T11 can be used to strengthen connections to the kidneys. In this instance the location of the neurological lesion is primarily in the brain and enteric nervous system. A physician applies a chosen modality to activate the T11 dermatome in this instance.

The dermatome for T12 can be used to strengthen connections to the kidneys. In this instance the location of the neurological lesion is primarily in the brainstem and enteric nervous system. A physician applies a chosen modality to activate the T12 dermatome in this instance.

The dermatome for L1 can be used to strengthen connections to the ileocecal valve. In this instance the location of the neurological lesion is primarily in the celiac ganglion, enteric nervous system, or brainstem. A physician applies a chosen modality to activate the L1 dermatome in this instance.

The dermatome for L2 can be used to strengthen connections to the celiac ganglion, superior mesenteric ganglion, and enteric nervous system. In this instance the location of the neurological lesion is primarily in the. A physician applies a chosen modality to activate the L2 dermatome in this instance.

The dermatome for L3 can be used to strengthen connections to the glandular system. In this instance the location of the neurological lesion is primarily in the hemispheric microglia or celiac ganglion. A physician applies a chosen modality to activate the L3 dermatome in this instance.

The dermatome for L4 can be used to strengthen connections to the colon. In this instance the location of the neurological lesion is primarily in the nucleus solitarius brainstem or liver ganglia. A physician applies a chosen modality to activate the L4 dermatome in this instance.

The dermatome for L5 can be used to strengthen connections to the gonads. In this instance the location of the neurological lesion is primarily in the hypothalamus or pituitary axis. A physician applies a chosen modality to activate the L5 dermatome in this instance.

The dermatome for S1 can be used to strengthen connections to the gallbladder and gastric system. In this instance the location of the neurological lesion is primarily in the celiac ganglion, enteric nervous system, or brainstem. A physician applies a chosen modality to activate the S1 dermatome in this instance.

The dermatome for S2 can be used to strengthen connections to the pancreas and cecum. In this instance the location of the neurological lesion is primarily in the celiac ganglion, superior mesenteric ganglion, or the enteric nervous system. A physician applies a chosen modality to activate the S2 dermatome in this instance.

The dermatome for S3 can be used to strengthen connections to the rectal sphincter. In this instance the location of the neurological lesion is primarily in the frontal lobe or pelvic plexus. A physician applies a chosen modality to activate the S3 dermatome in this instance.

The dermatome for S4 can be used to strengthen connections to the liver or colon. In this instance the location of the neurological lesion is primarily in the nucleus solitarius brainstem or the liver ganglia. A physician applies a chosen modality to activate the S4 dermatome in this instance.

The dermatome for S5 can be used to strengthen connections to the perianal area. In this instance the location of the neurological lesion is primarily in the frontal lobe or pelvic plexus. A physician applies a chosen modality to activate the S5 dermatome in this instance.

The physician can choose a selected modality to activate each dermatome. The chosen modality may be any type of stimuli applied to the surface of a dermatome. The chosen modality may activate mechanoreceptors, thermoreceptors, nocireceptors, or otherwise activate the firing of nerves from the chosen dermatome.

In one modality, the physician may choose to activate mechanoreceptors in the dermatome by applying physical pressure, vibration, or stretch stimuli. For instance, a physician can use a reflex hammer, such as that disclosed in U.S. Pat. No. 3,515,125 (Ruskin), U.S. Pat. No. 4,324,261 (Mark et al.), or U.S. Pat. No. 5,657,763 (Schneider) each of which is hereby fully incorporated by reference, to apply pressure to the chosen dermatome. The physician may also use the reflex hammer to stretch the patient's skin at the chosen dermatome. The physician may use the reflex hammer to manually apply vibrations to the chosen dermatome. Alternatively, the physician may use a tuning fork, such as that disclosed in U.S. Pat. No. 1,880,923 (Eisenhour), which is hereby fully incorporated by reference, to apply pressure or vibrations to the selected dermatome. Alternatively, the physician may perform these actions utilizing a stylus of a chiropractic adjusting instrument, also known as a chiropractic activator, such as that disclosed in U.S. D269,812 (Lancellotti) or U.S. Pat. No. 6,379,375 (Fuhr) each of which is hereby fully incorporated by reference. Alternatively, the physician may utilize an electrical vibration device, known as an ArthroStim device, such as that disclosed in U.S. Pat. No. 4,841,955 (Evans et al.) or U.S. Pat. No. 6,228,042 (Dungan) each of which is hereby full incorporated by reference, to apply vibration stimuli to the chosen dermatome. The electrical vibration device has a pressure sensitive stylus which vibrates longitudinally. The electrical vibration device produces a rapid recoiling of the stylus. The electrical vibration device activates the patients' proprioceptors and mechanoreceptors without recruiting the nocioceptors.

In another modality, the physician may choose to activate thermoreceptors in the dermatome by applying heat or a cold press to the patient's skin surface. For instance, the physician could activate the thermoreceptors of a chosen dermatome with heat by applying a heating pad to the patient's skin. A physician may also use cold to activate the thermoreceptors by applying ice or a cold press to the patient's skin.

In another modality, the physician may choose to activate nociceptors in the dermatome by presenting pain inducing stimuli to the patient's skin surface. The physician may do this by applying a sharp point of a needle to the patient's skin. In addition, the physician may apply a pinwheel, such as that disclosed in U.S. Pat. No. 5,433,212 (Greenfield) which is hereby fully incorporated by reference, to the patient's skin at the chosen dermatome.

In another modality, the physician may apply an electrical microcurrent to the dermatome. The electrical microcurrent activates the firing of nerves from the dermatome. The physician may utilize a transcutaneous electrical nerve stimulation unit (TENS unit), such as that disclosed in U.S. Pat. No. 4,989,605 (Rossen) which is hereby fully incorporated by reference, to activate the neurons in a chosen dermatome. In this modality the physician would apply two or more electrodes to the patient's skin at the chosen dermatome. The TENS unit applies a high frequency series of electrical stimuli (greater than 50 Hz) at an intensity lower than that required to initiate motor contraction. Alternatively, the physician may choose to utilize a somatosensory evoked potential (SEP) unit and method, such as that disclosed in U.S. 20060276720 (McGinnis et al.) which is hereby fully incorporated by reference. In this modality the physician applies one or more stimulating electrodes on the chosen dermatome. The physician places one or more recording electrodes on the patient's head or spine. The physician then causes the stimulating electrodes to generate an electrical signal which travels from the stimulating electrodes through a patient's neural pathway and is captured by the recording electrodes.

These modalities target neurological pathways that are well intact (viable), which connect with dysfunctional regions. These viable pathways are then manipulated (non-surgically and non-pharmaceutically), through the various modalities applied to various myotomal and dermatomal regions, to strengthen connections with faulty regions, causing neuroplasticity and regulation and normalization of function.

The modalities operate to stimulate the neural network connected to the chosen dermatome. This stimulation produces neuroplasticity via synaptogenesis in the collateral nerve branches of the activated neural network. Through repeated application of the modalities to the chosen dermatome, the collateral branches of the neural pathway are strengthened, thereby bypassing any lesion blocking normal pathway processes.

The physician may apply any number or type of these modalities of treatment to the chosen dermatome(s). To ensure improvement of the neural network and that the modality has caused a bypass of the neural lesion, the physician repeats the initial testing. This repeated testing may include full testing or solely a repeated SEP test procedure. The repeated testing allows a physician to determine an improvement in the patient's position and ensure that the chosen modality is clinically effective.

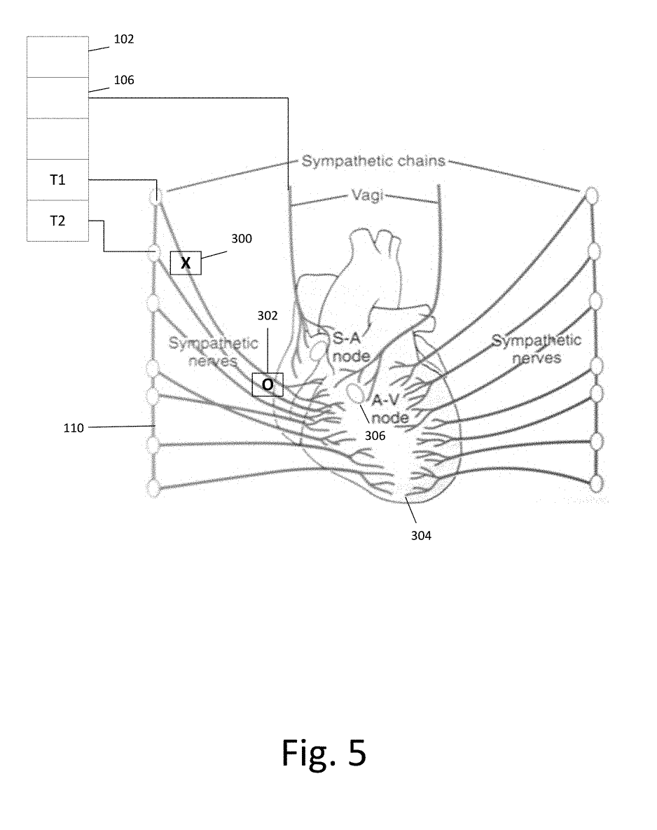

Referring to FIG. 5, an example of an application of the invention is illustrated. In this example a neural lesion 300 exists in a spinal nerve emanating from T1 and innervating the A-V node 306 of the heart 304. The neural lesion 300 may make itself known through a number of symptoms, such as arrhythmia. To compensate for this neural lesion 300, the physician applies a chosen modality to the T2 dermatome. Applying the modality to the T2 dermatome activates neuroplasticity via synaptogenesis between the T2 nerve and T1 nerve along collateral branches at a synaptogenesis point 302. Signals traveling along the T1 nerve can thus bypass the lesion 300 by traveling along the T2 nerve and crossing over at the synaptogenesis point 302. This bypass can resolve any symptoms presented by the patient.

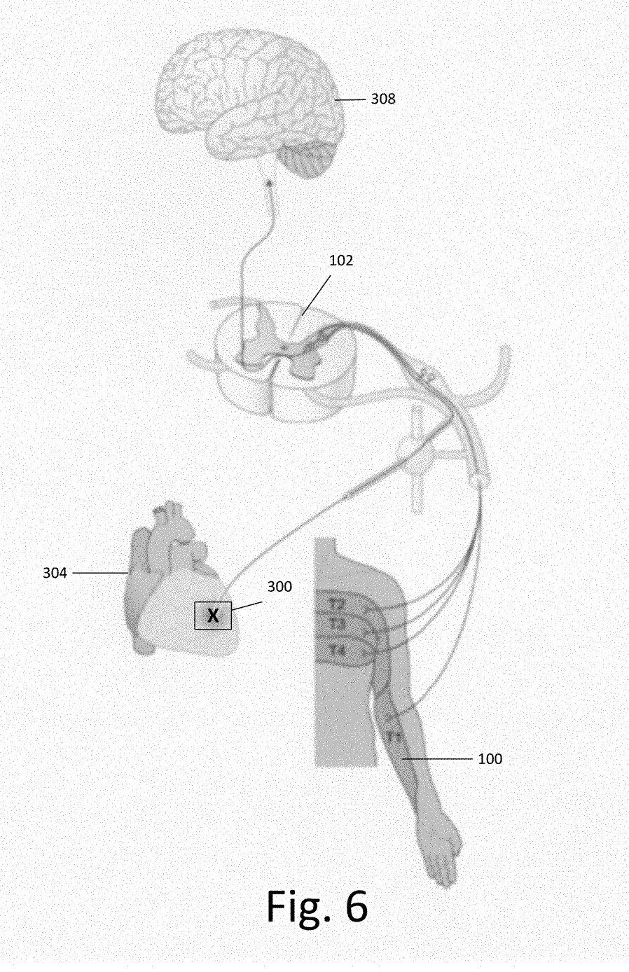

The example is further illustrated in FIG. 6 at the systems level. As shown in FIG. 6, the heart 304 has a neural lesion 300 at the connection of the spinal nerve. The spinal nerve carries somatosensory information from the heart 304, through the spinal cord 102, and into the brain 308. Based on information received by the brain 308, the brain 308 will send a motor signal to the heart 304, controlling the actions of the heart. If the brain 308 is not receiving correct information about the status of the heat 304 due to the neural lesion 300, then the brain 308 is sending a motor signal to control the heart 304 that is incorrect. For instance, due to the neural lesion 300 the brain may believe that the heart 304 is beating slower than it is in reality and therefore send a motor signal to increase heart rate when an increase is not needed. Such a motor signal would therefore lead to arrhythmia in the patient. The inventive method seeks to cause integration in the brain 308 by changing the sensory signals received by the brain 308. In this instance, a physician applies a chosen modality to a selected dermatome 100. The modality causes a somatosensory signal to be sent from the dermatome 100, through the spinal cord 102, and to the brain 308. Because the somatosensory signal from the dermatome 100 is received by the brain 308 in the same manner as that from the heart 304, the brain bases its motor signal response based on the combination of signals-essentially interpreting the somatosensory signal from the dermatome 100 as having come from the heart 308. By applying the chosen modality to the dermatome 100, the brain 308 is now integrating all sensory input and resetting the motor signal it generates, thereby correcting for deficiencies created by the neural lesion 300.

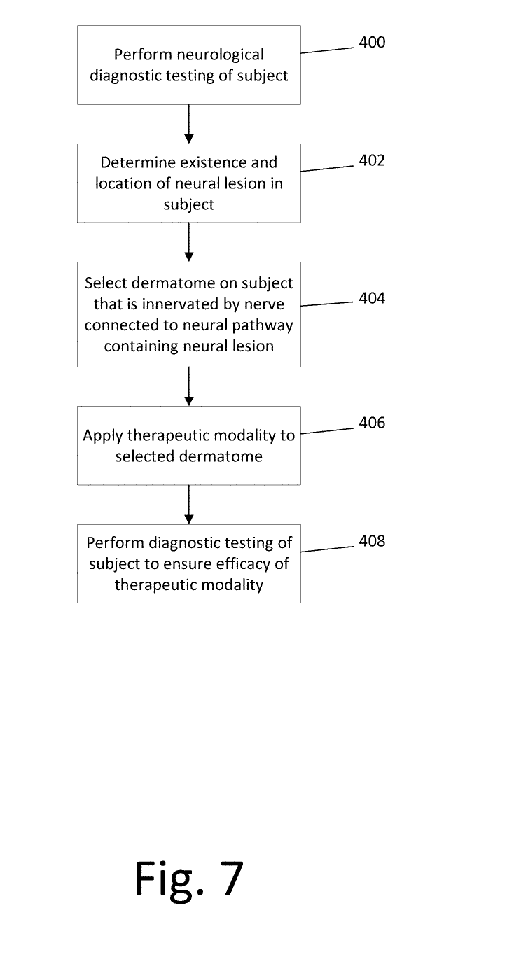

The method of the invention is illustrated by FIG. 7. First the physician performs neurological diagnostic testing of the subject 400. Then the physician determines the existence and the location of a neural lesion in the subject 402. The physician selects a dermatome on the subject that is innervated by a nerve that is connected to a neural pathway containing the neural lesion 404. The physician then applies a therapeutic modality to the selected dermatome 406. The physician may then perform diagnostic testing of the subject to ensure efficacy of the therapeutic modality 408.

The inventive methodology can be used to treat the causes of addiction. Standard models treat addiction in a number of ways but primarily target the symptoms of addition rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of addition.

The inventive methodology can be used to treat the causes of ADHD. Standard models treat ADHD in a number of ways but primarily target the symptoms of ADHD rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of ADHD.

The inventive methodology can be used to treat the causes of Alzheimer's. Standard models treat Alzheimer's in a number of ways but primarily target the symptoms of Alzheimer's rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of Alzheimer's.

The inventive methodology can be used to treat the causes of ALS. Standard models treat ALS in a number of ways but primarily target the symptoms of ALS rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of ALS.

The inventive methodology can be used to treat the causes of anxiety. Standard models treat anxiety in a number of ways but primarily target the symptoms of anxiety rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of anxiety.

The inventive methodology can be used to treat the causes of autism. Standard models treat autism in a number of ways but primarily target the symptoms of autism rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of autism.

The inventive methodology can be used to treat the causes of balance disorders. Standard models treat balance disorders in a number of ways but primarily target the symptoms of balance disorders rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of balance disorders.

The inventive methodology can be used to treat the causes of breathing disorders. Standard models treat breathing disorders in a number of ways but primarily target the symptoms of breathing disorders rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of breathing disorders.

The inventive methodology can be used to treat the causes of carpal tunnel. Standard models treat carpal tunnel in a number of ways but primarily target the symptoms of carpal tunnel rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of carpal tunnel.

The inventive methodology can be used to treat the causes of celiac disease. Standard models treat celiac disease in a number of ways but primarily target the symptoms of celiac disease rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of celiac disease.

The inventive methodology can be used to treat the causes of cerebral palsy. Standard models treat cerebral palsy in a number of ways but primarily target the symptoms of cerebral palsy rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of cerebral palsy.

The inventive methodology can be used to treat the causes of chronic fatigue. Standard models treat chronic fatigue in a number of ways but primarily target the symptoms of chronic fatigue rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of chronic fatigue.

The inventive methodology can be used to treat the causes of chronic neck and back pain. Standard models treat chronic neck and back pain in a number of ways but primarily target the symptoms of chronic neck and back pain rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of chronic neck and back pain.

The inventive methodology can be used to treat the causes of dangerous diet. Standard models treat dangerous diet in a number of ways but primarily target the symptoms of dangerous diet rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of dangerous diet.

The inventive methodology can be used to treat the causes of diabetes. Standard models treat diabetes in a number of ways but primarily target the symptoms of diabetes rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of diabetes.

The inventive methodology can be used to treat the causes of dysauntonomia. Standard models treat dysauntonomia in a number of ways but primarily target the symptoms of dysauntonomia rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of dysautonomia.

The inventive methodology can be used to treat the causes of dystonia. Standard models treat dystonia in a number of ways but primarily target the symptoms of dystonia rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of dystonia.

The inventive methodology can be used to treat the causes of esophageal disorders. Standard models treat esophageal disorders in a number of ways but primarily target the symptoms of esophageal disorders rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of esophageal disorders.

The inventive methodology can be used to treat the causes of fibromyalgia. Standard models treat fibromyalgia in a number of ways but primarily target the symptoms of fibromyalgia rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of fibromyalgia.

The inventive methodology can be used to treat the causes of headaches. Standard models treat headaches in a number of ways but primarily target the symptoms of headaches rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of headaches.

The inventive methodology can be used to treat the causes of homeostasis. Standard models treat homeostasis in a number of ways but primarily target the symptoms of homeostasis rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of homeostasis.

The inventive methodology can be used to treat the causes of hypertension. Standard models treat hypertension in a number of ways but primarily target the symptoms of hypertension rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of hypertension.

The inventive methodology can be used to treat the causes of hyperthyroidism. Standard models treat hyperthyroidism in a number of ways but primarily target the symptoms of hyperthyroidism rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of hyperthyroidism.

The inventive methodology can be used to treat the causes of hypothyroidism. Standard models treat hypothyroidism in a number of ways but primarily target the symptoms of hypothyroidism rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of hypothyroidism.

The inventive methodology can be used to treat the causes of inflammatory bowel disease. Standard models treat inflammatory bowel disease in a number of ways but primarily target the symptoms of inflammatory bowel disease rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of inflammatory bowel disease.

The inventive methodology can be used to treat the causes of kidney disease. Standard models treat kidney disease in a number of ways but primarily target the symptoms of kidney disease rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of kidney disease.

The inventive methodology can be used to treat the causes of liver disease. Standard models treat liver disease in a number of ways but primarily target the symptoms of liver disease rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of liver disease.

The inventive methodology can be used to treat the causes of multiple sclerosis. Standard models treat multiple sclerosis in a number of ways but primarily target the symptoms of multiple sclerosis rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of multiple sclerosis.

The inventive methodology can be used to treat the causes of Parkinson's disease. Standard models treat Parkinson's disease in a number of ways but primarily target the symptoms of Parkinson's disease rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of Parkinson's disease.

The inventive methodology can be used to treat the causes of peripheral. Standard models treat peripheral in a number of ways but primarily target the symptoms of peripheral rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of peripheral.

The inventive methodology can be used to treat the causes of psoriasis. Standard models treat psoriasis in a number of ways but primarily target the symptoms of psoriasis rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of psoriasis.

The inventive methodology can be used to treat the causes of restless leg syndrome. Standard models treat restless leg syndrome in a number of ways but primarily target the symptoms of restless leg syndrome rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of restless leg syndrome.

The inventive methodology can be used to treat the causes of stoke. Standard models treat stroke in a number of ways but primarily target the symptoms of stroke rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of stroke.

The inventive methodology can be used to treat the causes of tremor. Standard models treat tremor in a number of ways but primarily target the symptoms of tremor rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of tremor.

The inventive methodology can be used to treat the causes of vascular disease. Standard models treat vascular disease in a number of ways but primarily target the symptoms of vascular disease rather than the underlying cause. Prolonged drug use can lead to systemic inflammation or hepatic encephalopathy. The use leads to an accumulation of symptoms, each of which is treated separately under the standard model of health care. The incentive method treats the underlying neurological, psychological, and chemical processes of the body that are not addressed by these standard methods of treatment. The inventive method normalizes the nervous system, restoring brain and nerve function to normal, thereby eliminating the underlying cause of vascular disease.

What has been described above includes examples of the claimed subject matter. It is, of course, not possible to describe every conceivable combination of components or methodologies for purposes of describing the claimed subject matter, but one of ordinary skill in the art can recognize that many further combinations and permutations of such matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

1) A method for treating a neural lesion comprising
a) determining the existence of a neural lesion in a subject;
b) selecting a dermatome on said subject
i) wherein said dermatome is innervated by a nerve connected to a neural pathway containing said neural lesion;
c) applying a therapeutic modality to said dermatome
i) wherein said therapeutic modality is sufficient to activate neuroplasticity via synaptogenesis within collateral branches of said neural pathway.

2) The method as in claim 1 wherein said dermatome is selected from a group consisting of: C2, C3, C4, C5, T1, T2, T3, T4, T5, T6, T7, T8, T9, T10, T11, T12, L1, L2, L3, L4, L5, S1, S2, S3, S4, and S5.

3) The method as in claim 1 wherein said therapeutic modality is selected from a group consisting of:
a) applying consistent pressure over a predetermined period of time with a reflex hammer;
b) applying a rhythmic vibrational force over a predetermined period of time with a reflex hammer;
c) applying a force over a predetermined period of time with a tuning fork;
d) stretching a portion of skin with a stylus of a chiropractic adjusting instrument;
e) applying consistent pressure over a predetermined period of time with a stylus of a chiropractic adjusting instrument;
f) applying a rhythmic vibrational force over a predetermined period of time with a stylus of a chiropractic adjusting instrument;
g) stretching a portion of skin with a stylus of a chiropractic adjusting instrument; and
h) applying a rhythmic vibrational force over a predetermined period of time with a stylus of an electrical vibrational device.

4) The method as in claim 1 wherein said therapeutic modality is selected from a group consisting of:
a) applying an object to a portion of skin of said subject, said object having a temperature greater than a temperature of said portion of skin of said subject; and
b) applying an object to a portion of skin of said subject, said object having a temperature less than a temperature of said portion of skin of said subject.

5) The method as in claim 1 wherein said therapeutic modality is selected from a group consisting of:
a) poking a portion of skin of said subject with a needle; and
b) rolling a pinwheel on a portion of skin of said subject for a predetermined period of time.

6) The method as in claim 1 wherein said therapeutic modality is selected from a group consisting of:
a) applying an electrical current to a portion of skin of said subject by means of a transcutaneous electrical nerve stimulation unit;
b) applying two or more electrodes of a transcutaneous electrical nerve stimulation device to a portion of skin of said subject and generating a low intensity electrical impulse having a frequency of greater than 50 Hz from said two or more electrodes; and
c) performing a somatosensory evoked potential test comprising
i) placing one or more stimulating electrodes on a chosen dermatome on said subject;
ii) placing one or more recording electrodes on the cranium of said subject;
iii) causing said one or more stimulating electrodes to generate an electrical impulse which is received by said one or more recording electrodes; and
iv) recording data received by said one or more recording electrodes.

7) The method as in claim 1 further comprising performing a somatosensory evoked potential (SEP) test on said subject prior to applying said therapeutic modality.

8) The method as in claim 7 wherein performing a somatosensory evoked potential test comprises
a) placing one or more stimulating electrodes on a chosen dermatome on said subject;
b) placing one or more recording electrodes on the cranium of said subject;
c) causing said one or more stimulating electrodes to generate an electrical impulse which is received by said one or more recording electrodes; and
d) recording data received by said one or more recording electrodes.

9) The method as in claim 1 further comprising performing a second somatosensory evoked potential (SEP) test on said subject after applying said therapeutic modality to ensure efficacy of said therapeutic modality.

10) The method as in claim 9 wherein performing a second somatosensory evoked potential test comprises
a) placing one or more stimulating electrodes on a chosen dermatome on said subject;
b) placing one or more recording electrodes on the cranium of said subject;
c) causing said one or more stimulating electrodes to generate an electrical impulse which is received by said one or more recording electrodes; and
d) recording data received by said one or more recording electrodes.

11) The method as in claim 2 wherein said therapeutic modality is selected from a group consisting of:
a) applying consistent pressure over a predetermined period of time with a reflex hammer;
b) applying a rhythmic vibrational force over a predetermined period of time with a reflex hammer;
c) applying a force over a predetermined period of time with a tuning fork;
d) stretching a portion of skin with a stylus of a chiropractic adjusting instrument;
e) applying consistent pressure over a predetermined period of time with a stylus of a chiropractic adjusting instrument;
f) applying a rhythmic vibrational force over a predetermined period of time with a stylus of a chiropractic adjusting instrument;
g) stretching a portion of skin with a stylus of a chiropractic adjusting instrument;
h) applying a rhythmic vibrational force over a predetermined period of time with a stylus of an electrical vibrational device;
i) applying an object to a portion of skin of said subject, said object having a temperature greater than a temperature of said portion of skin of said subject;
j) applying an object to a portion of skin of said subject, said object having a temperature less than a temperature of said portion of skin of said subject;
k) poking a portion of skin of said subject with a needle;
l) rolling a pinwheel on a portion of skin of said subject for a predetermined period of time;
m) applying an electrical current to a portion of skin of said subject by means of a transcutaneous electrical nerve stimulation unit;
n) applying two or more electrodes of a transcutaneous electrical nerve stimulation device to a portion of skin of said subject and generating a low intensity electrical impulse having a frequency of greater than 50 Hz from said two or more electrodes; and
o) performing a somatosensory evoked potential test comprising
i) placing one or more stimulating electrodes on a chosen dermatome on said subject;
ii) placing one or more recording electrodes on the cranium of said subject;
iii) causing said one or more stimulating electrodes to generate an electrical impulse which is received by said one or more recording electrodes; and
iv) recording data received by said one or more recording electrodes.

12) The method as in claim 11 further comprising performing a somatosensory evoked potential (SEP) test on said subject prior to applying said therapeutic modality.

13) The method as in claim 12 wherein performing a somatosensory evoked potential test prior to applying said therapeutic modality comprises
a) placing one or more stimulating electrodes on a chosen dermatome on said subject;
b) placing one or more recording electrodes on the cranium of said subject;
c) causing said one or more stimulating electrodes to generate an electrical impulse which is received by said one or more recording electrodes; and
d) recording data received by said one or more recording electrodes.

14) The method as in claim 13 further comprising performing a second somatosensory evoked potential (SEP) test on said subject after applying said therapeutic modality to ensure efficacy of said therapeutic modality.

15) The method as in claim 14 wherein performing a second somatosensory evoked potential test comprises
a) placing one or more stimulating electrodes on a chosen dermatome on said subject;
b) placing one or more recording electrodes on the cranium of said subject;
c) causing said one or more stimulating electrodes to generate an electrical impulse which is received by said one or more recording electrodes; and
d) recording data received by said one or more recording electrodes.
